(19) United States
(12) Patent Application Publication
(10) Pub. No.: US 2025/0281640 A1
(43) Pub. Date: Sep. 11, 2025

(54) NUCLEIC ACID HAVING PROMOTER ACTIVITY AND USE THEREOF

(71) Applicant: JOINT STOCK COMPANY "BIOCAD", Saint Petersburg (RU)

(72) Inventors: Elena Veniaminovna VLASOVA, g. Sortavala (RU); Anna Nikolaevna STRELKOVA, g. Yaransk (RU); Konstantin Ilich TYSHCHUK, Saint Petersburg (RU); Pavel Mikhailovich GERSHOVICH, Saint Petersburg (RU); Mariya Pavlovna PEREPELKINA, Saint Petersburg (RU); Pavel Andreevich IAKOVLEV, Saint Petersburg (RU); Dmitry Valentinovich MOROZOV, Saint Petersburg (RU)

(21) Appl. No.: 18/863,824

(22) PCT Filed: Jun. 11, 2023
(86) PCT No.: PCT/RU2023/050148

§ 371 Date: Nov. 7, 2024

(30) Foreign Application Priority Data
Jun. 10, 2022 (RU) 2022115853

(51) Int. Cl. A61K 48/00; A61K 38/37; C12N 15/86

(52) U.S. Cl. CPC A61K 48/005; A61K 38/37; C12N 15/86; C12N 2750/14143

(57) ABSTRACT

The present application relates to the fields of genetics, gene therapy, and molecular biology. More specifically, the present invention relates to a nucleic acid having promoter activity (variants), an expression cassette and a vector based thereon, a host cell for producing a target product or expression vector.

### FIELD OF THE INVENTION

The present application relates to the fields of genetics, gene therapy, and molecular biology. More specifically, the present invention relates to a nucleic acid having promoter activity (variants), an expression cassette and a vector based thereon, a host cell for producing a target product or expression vector.

### BACKGROUND OF THE INVENTION

A promoter is a DNA element which promotes transcription of a polynucleotide to which the promoter is operably linked. The promoter may also be part of a promoter/enhancer element. Although the physical boundaries between the promoter and enhancer elements are not always clear, the promoter typically refers to a site on a nucleic acid molecule to which an RNA polymerase and/or any associated factors binds and at which transcription is initiated. Enhancers potentiate promoter activity temporally as well as spatially. Many promoters are known to be transcriptionally active in a wide range of cell types. Promoters may be divided into two classes, those that function constitutively and those that are regulated by induction or derepression. The both classes are suitable for protein expression. Promoters that are used for high-level production of polypeptides in eukaryotic cells and, in particular, in mammalian cells, should be strong and preferably active in a wide range of cell types. Strong constitutive promoters which are capable of driving expression in many cell types are well known in the art and, therefore, it is not herein necessary to describe them in detail.

Examples of promoters and/or enhancers are promoters and/or enhancers derived from retroviral LTR, cytomegalovirus (CMV) (for example, CMV promoter/enhancer), simian virus 40 (SV40) (for example, SV40 promoter/enhancer), adenovirus, (for example, adenovirus major late promoter (AdMLP)), CAG promoter, as well as strong mammalian promoters such as TTR promoter or EF1-a promoter.

The CMV promoter, depending on the subtype, has a length of 589 to 1650 bp (Changyu Zheng ET ALL., All Human EF1α Promoters Are Not Equal: Markedly Affect Gene Expression in Constructs from Different Sources, Int J Med Sci. 2014; 11 (5): 404-408, doi: 10.7150/ijms.8033). The CAG promoter (CMV early enhancer/chicken β actin) has a length of 868 bp (Nieuwenhuis, B., Haenzi, B., Hilton, S. et al. Optimization of adeno-associated viral vector-mediated transduction of the corticospinal tract: comparison of four promoters. Gene Ther. 2021; 28:56-74, https://doi.org/10.1038/41434-020. 0169-1).

All of the above promoters have a length over 588 bp. Promoters with a length over 588 bp in expression cassettes are not the most optimal options for the expression of large transgenes using a number of expression vectors.

Thus, there is a need to create short promoters.

Many promoters, including the CMV promoter, are not tissue-specific, i.e., they do not provide for selective expression of therapeutic transgenes in cells of certain organs, which fact is an apparent disadvantage of these promoters when used to create a gene therapy product.

Thus, there is a need to create tissue-specific promoters that provide for selective expression of therapeutic transgenes in cells of certain organs.

### DISCLOSURE OF THE ESSENCE OF THE INVENTION

The authors of the invention have surprisingly found that a nucleic acid having a nucleotide sequence that is selected from the group comprising SEQ ID NO: 1, 2, 3, 4, 5, 6, 7, 8, 9, 10, 11, 12, 13, 14 or 15 has promoter activity. The promoters having the nucleotide sequence of SEQ ID NO:1, 2, 3, 4, 5, 6, 7, 8, 9, 10, 11, 12, 13, 14 or 15 have a length ranging from 123 to 252 bp. Furthermore, the authors of the invention have surprisingly found that promoters having the nucleotide sequence of SEQ ID NO: 1, 2, 3, 4, 5, 6, 7, 8, 9, 10, 11, 12, 13, 14 or 15 have tissue-specific promoter activity in cells of hepatocytic origin.

### Definitions and General Methods

Unless defined otherwise herein, all technical and scientific terms used in connection with the present invention will have the same meaning as is commonly understood by those skilled in the art.

Furthermore, unless otherwise required by context, singular terms shall include plural terms, and the plural terms shall include the singular terms. Typically, the present classification and methods of cell culture, molecular biology, immunology, microbiology, genetics, analytical chemistry, organic synthesis chemistry, medical and pharmaceutical chemistry, as well as hybridization and chemistry of protein and nucleic acids described herein are well known by those skilled and widely used in the art. Enzyme reactions and purification methods are performed according to the manufacturer's guidelines, as is common in the art, or as described herein.

As used in the present description and claims that follow, unless otherwise dictated by the context, the words “include” and “comprise”, or variations thereof such as “includes”, “including”, “comprises”, or “comprising”, will be understood to imply the inclusion of a stated integer or group of integers but not the exclusion of any other integer or group of integers.

### Nucleic Acid Molecules

The terms “nucleic acid”, “nucleic sequence”, “nucleic acid sequence”, “polynucleotide”, “oligonucleotide”, “polynucleotide sequence” and “nucleotide sequence”, used interchangeably in the present description, mean a precise sequence of nucleotides, modified or not, determining a fragment or a region of a nucleic acid, containing unnatural nucleotides or not, and being either a double-strand DNA or RNA, a single-strand DNA or RNA, or transcription products of said DNAs.

As used in the present description, polynucleotides include, by way of non-limiting examples, all nucleic acid sequences which are obtained by any means available in the art, including, as non-limiting examples, recombinant means, i.e. the cloning of nucleic acid sequences from a recombinant library or a cell genome, using ordinary cloning technology and PCR and the like, and by synthetic means.

It should also be included here that the present invention does not relate to nucleotide sequences in natural chromosomal environment thereof, i.e. in a natural state. The sequences of the present invention have been isolated and/or purified, i.e., they were sampled directly or indirectly, for example by way of copying, whereby the environment thereof have been at least partially modified. Thus, isolated nucleic acids obtained by way of recombinant genetics, for example, using host cells, or obtained by way of chemical synthesis should also be mentioned here.

Unless otherwise indicated, the term nucleotide sequence encompasses its complement. Thus, a nucleic acid having a particular sequence should be understood as one which encompasses the complementary strand thereof with the complementary sequence thereof.

### Vector

The term “vector” as used herein means a nucleic acid molecule capable of transporting another nucleic acid to which it has been linked. Furthermore, the term “vector” herein refers to a recombinant viral particle capable of transporting a nucleic acid.

As used in the present description, the term “expression” is defined as the transcription and/or translation of a particular nucleotide sequence driven by its promoter.

### DETAILED DESCRIPTION OF THE INVENTION

### Nucleic Acid

In one aspect, the present invention relates to a nucleic acid that has promoter activity and includes a nucleotide sequence that is selected from the group of SEQ ID NO: 1, 2, 3, 4, 5, 6, 7, 8, 9, 10, 11, 12, 13, 14 or 15.

In some embodiments of the invention, the nucleic acid is an isolated nucleic acid.

An “isolated” nucleic acid molecule is one which is identified and separated from at least one nucleic acid molecule-impurity. An isolated nucleic acid molecule is different from the form or set in which it is found under natural conditions. Thus, an isolated nucleic acid molecule is different from a nucleic acid molecule that exists in cells under natural conditions.

All nucleic acids having the nucleotide sequence of SEQ ID NO:1, 2, 3, 4, 5, 6, 7, 8, 9, 10, 11, 12, 13, 14 or 15 have promoter activity.

All promoters having the nucleotide sequence of SEQ ID NO:1, 2, 3, 4, 5, 6, 7, 8, 9, 10, 11, 12, 13, 14 or 15 have tissue-specific promoter activity in cells of hepatocytic origin.

All promoters having the nucleotide sequence of SEQ ID NO:1, 2, 3, 4, 5, 6, 7, 8, 9, 10, 11, 12, 13, 14 or 15 are strong promoters and the use thereof as part of expression vectors leads to increased levels of production and activity of the target protein.

Expression Cassette. Expression Vector.

In one aspect, the present invention relates to an expression cassette comprising any of the above nucleic acids having promoter activity.

The term “cassette which expresses” or “expression cassette”, as used herein, refers in particular to a DNA fragment that is capable, in an appropriate setting, of triggering the expression of a polynucleotide encoding a polypeptide of interest, the sequence of which is included in said expression cassette. When introduced into a host cell, the expression cassette is, inter alia, capable of engaging cellular mechanisms to transcribe the polynucleotide encoding the polypeptide of interest into RNA which is then typically further processed and finally translated into the polypeptide of interest. The expression cassette may be contained in an expression vector.

In some embodiments, the expression cassette includes the following elements in the 5′-end to 3′-end direction:
a left-hand (first) ITR (inverted terminal repeats);
any one of the above nucleic acids having promoter activity;
a transgene;
a polyadenylation signal;
a right-hand (second) ITR.

The above structural elements of the expression cassette are operably linked to one another.

As used herein, the term “operably linked” refers to a linkage of polynucleotide (or polypeptide) elements into a functional relationship. A nucleic acid is “operably linked” when it is present in functional relationship conditions with another nucleic acid sequence. For example, a transcription regulatory sequence is operably linked to a coding sequence if it affects the transcription of said coding sequence. The term “operably linked” means that the linked DNA sequences are typically contiguous and, where it is necessary to join two protein encoding regions, are also contiguous and are present in the reading frame.

In some embodiments, the expression cassette includes a left-hand (first) ITR with the nucleotide sequence of SEQ ID NO: 16.

In some embodiments, the expression cassette includes a polyadenylation signal with the nucleotide sequence of SEQ ID NO: 17.

In some embodiments, the expression cassette includes a right-hand (second) ITR with the nucleotide sequence of SEQ ID NO: 18.

In some embodiments, the expression cassette includes the following elements in the 5′-end to 3′-end direction:
a left-hand (first) ITR (inverted terminal repeats) with the nucleotide sequence of SEQ ID NO: 16;
a promoter having a nucleotide sequence selected from the group of SEQ ID NO:1, 2, 3, 4, 5, 6, 7, 8, 9, 10, 11, 12, 13, 14 or 15;
a transgene;
a polyadenylation signal with the nucleotide sequence of SEQ ID NO: 17;
a right-hand (second) ITR with the nucleotide sequence of SEQ ID NO: 18.

In some embodiments, the expression cassette includes a transgene that encodes a protein or a small inhibitory nucleic acid.

In some embodiments, the expression cassette includes a transgene that encodes a protein that is selected from a group comprising factor VIII or a functional variant thereof, factor IX or a functional variant thereof, SMN1 protein (survival motor neuron protein), the RBD-S polypeptide of SARS-cov2, or a therapeutic antibody.

In one aspect, the present invention relates to an expression vector that includes any of the above nucleic acids having promoter activity or any of the above expression cassettes.

In some embodiments of the invention, the vectors are capable of autonomous replication in a host cell into which they are introduced (e.g. bacterial vectors having a bacterial site of replication origin and episomal vectors). In further embodiments of the invention, the vectors (e.g. non-episomal vectors) may be integrated into the genome of a host cell upon introduction into a host cell, and thereby are replicated along with the host genome. Moreover, certain vectors are capable of directing the expression of genes to which they are operably linked. Such vectors are referred to herein as “recombinant expression vectors” (or simply, “expression vectors”).

Expression vectors include plasmids, retroviruses, adenoviruses, adeno-associated viruses (AAVs), plant viruses, such as cauliflower mosaic virus, tobacco mosaic virus, cosmids, YACs, EBV, and the like. DNA molecules may be inserted into a vector such that transcriptional and translational control sequences within the vector serve their intended function of regulating the transcription and translation of the DNA. An expression vector and expression control sequences may be chosen to be compatible with the expression host cell used. DNA molecules may be introduced into the expression vector by standard methods (e.g. ligation of complementary restriction sites, or blunt end ligation if no restriction sites are present).

In some embodiments of the invention, the vector is a plasmid, AAV, adenovirus or lentivirus.

In some embodiments of the invention, the vector is a plasmid, i.e. a circular double stranded DNA into which additional DNA segments may be inserted.

In some embodiments of the invention, the vector is a viral (expression) vector, wherein additional DNA segments may be inserted into the viral genome.

In some embodiments, the expression vector is a recombinant adeno-associated virus (AAV).

In some embodiments, the AAV is selected from a group including the following AAV serotypes: AAV1, AAV2, AAV3, AAV4, AAV5, AAV6, AAV7, AAV8, AAV9, AAV10 AAV11, AAV12, AAV13, AAV14, AAV15, AAV16, rAAV.rh8, rAAV.rhlO, rAAV.rh20, rAAV.rh39, rAAV.Rh74, rAAV.RHM4-1, AAV.hu37, rAAV.Anc80, rAAV.Anc80L65, rAAV.7m8, rAAV.PHP.B, rAAV2.5, rAAV2.6, rAAV2.8, rAAV2.9, rAAV2tYF, rAAV3B, rAAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15 or AAV.HSC16.

In some embodiments of the invention, the vector or cassette, apart from a promoter, may include other expression control sequence. The term “other expression control sequence” as used in the present description refers to polynucleotide sequences that are necessary to effect the expression and processing of coding sequences to which they are inserted. It will be understood by those skilled in the art that the design of an expression vector or cassette, including the selection of expression control sequences, may depend on such factors as the choice of the type of a host cell to be transformed, the required level of expression of a protein, and so forth. The expression control sequences, apart from a promoter, include corresponding transcription initiation, termination, and enhancer sequences; efficient RNA processing signals such as splicing and polyadenylation signals; sequences which stabilize cytoplasmic mRNA; sequences that enhance translation efficiency (i.e., Kozak consensus sequence); sequences which enhance protein stability; and when desired, sequences which enhance protein secretion. The nature of such control sequences differs depending upon the host organism; in prokaryotes, such control sequences generally include a promoter, a ribosome binding site, as well as transcription termination sequences; in eukaryotes, such control sequences typically include promoters and transcription termination sequences. Expression control sequences encompass at least all components whose presence is important for expression and processing.

In addition to the above genes and expression control sequences, the recombinant expression vectors according to the invention may carry additional sequences, such as sequences which regulate replication of the vector in host cells (e.g. origins of replication) and selectable marker genes. The selectable marker gene facilitates the selection of host cells into which the vector or cassette has been introduced.

In one embodiment of the present invention, the expression vector relates to a vector comprising one or more polynucleotide sequences of interest, genes of interest, or transgenes that are flanked by parvoviral sequences or inverted terminal repeat (ITR) sequences.

Neither the cassette nor the vector of the invention comprises nucleotide sequences of genes encoding non-structural proteins (Rep) and structural proteins (Cap) of the adeno-associated virus.

### Host Cell

In one aspect, the present invention relates to a host cell for producing a target product or for producing any one of the above expression vectors, which comprises any one of the above nucleic acids having promoter activity.

The term “host cell” as used herein refers to a cell into which the recombinant expression vector or cassette according to the invention has been introduced. It should be understood that “host cell” refers not only to a particular subject cell but to the progeny of such cell as well. Since modifications may occur in succeeding generations due to either mutation or environmental influences, such progeny may not, in fact, be identical to a parental cell; however, such cells are still included within the scope of the term “host cell” as used herein.

The expression vectors or cassettes according to the invention may be used for transfection of a mammalian cell, plant cell, bacterial or yeast host cell. Transfection may be carried out by any known method for introducing polynucleotides into a host cell. Methods for introducing heterologous polynucleotides into mammalian cells are well known in the art and include dextran-mediated transfection, cationic polymer-nucleic acid complex transfection, calcium phosphate precipitation, polybrene-mediated transfection, protoplast fusion, encapsulation of the polynucleotides in liposomes, and direct microinjection of DNA into nuclei. In addition, the nucleic acid molecules may be introduced into mammalian cells by viral (expression) vectors.

Mammalian cell lines used as hosts for transfection are well known in the art and include a plurality of immortalized cell lines available. These include, e.g., Chinese hamster ovary (CHO) cells, NS0 cells, SP2 cells, HEK-293T cells, FreeStyle 293 cells (Invitrogen), NIH-3T3 cells, HeLa cells, baby hamster kidney (BHK) cells, African green monkey kidney cells (COS), human hepatocellular carcinoma cells (e.g., Hep G2), A549 cells, SK-HEP1, HUH7, Hep-RG and a number of other cell lines. Cell lines are selected by way of determining which cell lines have high expression levels and provide for necessary characteristics of the protein being produced. Other cell lines that may be used are insect cell lines, such as Sf9 or Sf21 cells. When the recombinant expression vectors according to the invention are introduced into mammalian host cells, the target protein is produced by way of culturing the host cells for a period of time sufficient to express the target protein in the host cells, or, more preferably, secrete the target protein into the culture medium in which the host cells are cultured. The target protein may be isolated from culture medium using standard protein purification techniques. Plant host cells include e.g. Nicotiana, Arabidopsis, duckweed, corn, wheat, potato, etc. Bacterial host cells include Escherichia and Streptomyces species. Yeast host cells include Schizosaccharomyces pombe, Saccharomyces cerevisiae and Pichia pastoris.

The above host cell does not relate to a host cell produced using human embryos.

The above host cell does not relate to a host cell produced by modifying the genetic integrity of human germline cells.

### BRIEF DESCRIPTION OF DRAWINGS

FIG. 1 is a graph that shows increased proportion of fluorescence of cells expressing the EGFP reporter protein under control of promoters corresponding to the nucleic sequences of SEQ ID NO: 1 and 2.

Proportion of EGFP-expressing cells following transfection:
1—with an expression cassette with EGFP transgene and without a promoter region (control);
2—with an expression cassette with EGFP transgene and supplemented with cytomegalovirus (CMV) viral promoter region (control);

3—with an expression cassette with EGFP transgene under control of a promoter corresponding to the nucleic sequence of SEQ ID NO: 2;

4—with an expression cassette with EGFP transgene under control of a promoter corresponding to the nucleic sequence of SEQ ID NO: 1.

FIG. 2 is a graph that shows increased proportion of fluorescence of cells expressing the EGFP reporter protein under control of promoters corresponding to the nucleic sequences of SEQ ID NO: 3-7 and SEQ ID NO: 11-14.

Proportion of EGFP-expressing cells following transfection:
1—with an expression cassette with EGFP transgene and without a promoter region (control);
2—with an expression cassette with EGFP transgene under control of a promoter corresponding to the nucleic sequence of SEQ ID NO: 4;
3—with an expression cassette with EGFP transgene under control of a promoter corresponding to the nucleic sequence of SEQ ID NO: 5;
4—with an expression cassette with EGFP transgene under control of a promoter corresponding to the nucleic sequence of SEQ ID NO: 6;
5—with an expression cassette with EGFP transgene under control of a promoter corresponding to the nucleic sequence of SEQ ID NO: 7;
6—with an expression cassette with EGFP transgene under control of a promoter corresponding to the nucleic sequence of SEQ ID NO: 13;
7—with an expression cassette with EGFP transgene under control of a promoter corresponding to the nucleic sequence of SEQ ID NO: 14;
8—with an expression cassette with EGFP transgene under control of a promoter corresponding to the nucleic sequence of SEQ ID NO: 11;
9—with an expression cassette with EGFP transgene under control of a promoter corresponding to the nucleic sequence of SEQ ID NO: 12;
10—with an expression cassette with EGFP transgene under control of a promoter corresponding to the nucleic sequence of SEQ ID NO: 3.

FIG. 3 is a graph that shows increased proportion of fluorescence of cells expressing the EGFP reporter protein under control of promoters corresponding to the nucleic sequences of SEQ ID NO: 8-10 and 15.

Proportion of EGFP-expressing cells following transfection:
1—with an expression cassette with EGFP transgene and without a promoter region (control);
2—with an expression cassette with EGFP transgene under control of a promoter corresponding to the nucleic sequence of SEQ ID NO: 8;
3—with an expression cassette with EGFP transgene under control of a promoter corresponding to the nucleic sequence of SEQ ID NO: 10;
4—with an expression cassette with EGFP transgene under control of a promoter corresponding to the nucleic sequence of SEQ ID NO: 9;
5—with an expression cassette with EGFP transgene under control of a promoter corresponding to the nucleic sequence of SEQ ID NO: 15.

FIG. 4 is a graph that shows increased intensity of fluorescence of cells expressing the EGFP reporter protein under control of promoters corresponding to the nucleic sequences of SEQ ID NO: 3-7 and SEQ ID NO: 11-14.

Intensity of fluorescence of cells expressing EGFP following transfection:
1—with an expression cassette with EGFP transgene and without a promoter region (control);
2—with an expression cassette with EGFP transgene under control of a promoter corresponding to the nucleic sequence of SEQ ID NO: 4;
3—with an expression cassette with EGFP transgene under control of a promoter corresponding to the nucleic sequence of SEQ ID NO: 5;
4—with an expression cassette with EGFP transgene under control of a promoter corresponding to the nucleic sequence of SEQ ID NO: 6;
5—with an expression cassette with EGFP transgene under control of a promoter corresponding to the nucleic sequence of SEQ ID NO: 7;
6—with an expression cassette with EGFP transgene under control of a promoter corresponding to the nucleic sequence of SEQ ID NO: 13;
7—with an expression cassette with EGFP transgene under control of a promoter corresponding to the nucleic sequence of SEQ ID NO: 14;
8—with an expression cassette with EGFP transgene under control of a promoter corresponding to the nucleic sequence of SEQ ID NO: 11;
9—with an expression cassette with EGFP transgene under control of a promoter corresponding to the nucleic sequence of SEQ ID NO: 12;
10—with an expression cassette with EGFP transgene under control of a promoter corresponding to the nucleic sequence of SEQ ID NO: 3.

FIG. 5 is a graph that shows increased intensity of fluorescence of cells expressing the EGFP reporter protein under control of promoters corresponding to the nucleic sequences of SEQ ID NO: 8-10 and 15.

Intensity of fluorescence of cells expressing EGFP following transfection:
1—with an expression cassette with EGFP transgene and without a promoter region (control);
2—with an expression cassette with EGFP transgene under control of a promoter corresponding to the nucleic sequence of SEQ ID NO: 8;
3—with an expression cassette with EGFP transgene under control of a promoter corresponding to the nucleic sequence of SEQ ID NO: 10;
4—with an expression cassette with EGFP transgene under control of a promoter corresponding to the nucleic sequence of SEQ ID NO: 9;
5—with an expression cassette with EGFP transgene under control of a promoter corresponding to the nucleic sequence of SEQ ID NO: 15.

FIG. 6 is a graph that shows increased levels of production of the coagulation protein while using nucleic acids exhibiting promoter activity and corresponding to the sequences of SEQ ID NO: 3, 11 and 14.

Levels of production of the coagulation protein while transfection:
1—with an expression cassette with coagulation factor transgene and without a promoter region (control);
2—with an expression cassette in which the coagulation factor transgene is under control of a promoter corresponding to the nucleic sequence of SEQ ID NO: 3;
3—with an expression cassette in which the coagulation factor transgene is under control of a promoter corresponding to the nucleic sequence of SEQ ID NO: 11;
4—with an expression cassette in which the coagulation factor transgene is under control of a promoter corresponding to the nucleic sequence of SEQ ID NO: 14.

FIG. 7 is a graph that shows increased levels of activity of the coagulation protein while using nucleic acids exhibiting promoter activity and corresponding to the sequences of SEQ ID NO: 3, 11 and 14.

Levels of activity of the coagulation protein while transfection:
1—with an expression cassette with coagulation factor transgene and without a promoter region (control);
2—with an expression cassette in which the coagulation factor transgene is under control of a promoter corresponding to the nucleic sequence of SEQ ID NO: 3;
3—with an expression cassette in which the coagulation factor transgene is under control of a promoter corresponding to the nucleic sequence of SEQ ID NO: 11;
4—with an expression cassette in which the coagulation factor transgene is under control of a promoter corresponding to the nucleic sequence of SEQ ID NO: 14.

FIG. 8 is a graph of measurement of tissue specificity of newly produced sequences on the Huh7 and U87 cell lines, which shows increased proportion of cells expressing the EGFP reporter protein under control of promoters corresponding to the nucleic sequences of SEQ ID NO: 3-7 and SEQ ID NO: 11-14.

Proportion of EGFP-expressing cells following transfection:
1—with an expression cassette with EGFP transgene and without a promoter region (control);
2—with an expression cassette with EGFP transgene under control of a promoter corresponding to the nucleic sequence of SEQ ID NO: 4;
3—with an expression cassette with EGFP transgene under control of a promoter corresponding to the nucleic sequence of SEQ ID NO: 5;
4—with an expression cassette with EGFP transgene under control of a promoter corresponding to the nucleic sequence of SEQ ID NO: 6;
5—with an expression cassette with EGFP transgene under control of a promoter corresponding to the nucleic sequence of SEQ ID NO: 7;
6—with an expression cassette with EGFP transgene under control of a promoter corresponding to the nucleic sequence of SEQ ID NO: 13;
7—with an expression cassette with EGFP transgene under control of a promoter corresponding to the nucleic sequence of SEQ ID NO: 14;
8—with an expression cassette with EGFP transgene under control of a promoter corresponding to the nucleic sequence of SEQ ID NO: 11;
9—with an expression cassette with EGFP transgene under control of a promoter corresponding to the nucleic sequence of SEQ ID NO: 12;
10—with an expression cassette with EGFP transgene under control of a promoter corresponding to the nucleic sequence of SEQ ID NO: 3.

FIG. 9 is a graph of measurement of tissue specificity of newly produced sequences on the Huh7, HepG2 and HEK293 cell lines, which shows increased proportion of cells expressing the EGFP reporter protein under control of promoters corresponding to the nucleic sequences of SEQ ID NO: 7 and 11.

Proportion of EGFP-expressing cells following transfection:
1—with an expression cassette with EGFP transgene and without a promoter region (control);
2—with an expression cassette with EGFP transgene under control of a promoter corresponding to the nucleic sequence of SEQ ID NO: 7;
3—with an expression cassette with EGFP transgene under control of a promoter corresponding to the nucleic sequence of SEQ ID NO: 11.

FIG. 10 is a graph of measurement of tissue specificity of produced sequences on the CHO cell line, which shows increased intensity of fluorescence of cells expressing the EGFP reporter protein under control of promoters corresponding to the nucleic sequences of SEQ ID NO: 1 and 2.

Intensity of fluorescence of cells expressing EGFP following transfection:
1—with an expression cassette with EGFP transgene and without a promoter region (control);
2—with an expression cassette with EGFP transgene and supplemented with cytomegalovirus (CMV) viral promoter region (control);
3—with an expression cassette with EGFP transgene under control of a promoter corresponding to the nucleic sequence of SEQ ID NO: 2;
4—with an expression cassette with EGFP transgene under control of a promoter corresponding to the nucleic sequence of SEQ ID NO: 1.

FIG. 11 is a graph of measurement of tissue specificity of produced sequences on the HepG2 cell line, which shows increased intensity of fluorescence of cells expressing the EGFP reporter protein under control of promoters corresponding to the nucleic sequences of SEQ ID NO: 1 and 2.

Intensity of fluorescence of cells expressing EGFP following transfection:
1—with an expression cassette with EGFP transgene and without a promoter region (control);
2—with an expression cassette with EGFP transgene and supplemented with cytomegalovirus (CMV) viral promoter region (control);
3—with an expression cassette with EGFP transgene under control of a promoter corresponding to the nucleic sequence of SEQ ID NO: 2;
4—with an expression cassette with EGFP transgene under control of a promoter corresponding to the nucleic sequence of SEQ ID NO: 1.

FIG. 12 is a graph of measurement of tissue specificity of produced sequences on the Huh7 and U87 cell lines, which shows increased intensity of fluorescence of cells expressing the EGFP reporter protein under control of promoters corresponding to the nucleic sequences of SEQ ID NO: 3-7 and SEQ ID NO: 11-14.

Intensity of fluorescence of cells expressing EGFP following transfection:
1—with an expression cassette with EGFP transgene and without a promoter region (control);
2—with an expression cassette with EGFP transgene under control of a promoter corresponding to the nucleic sequence of SEQ ID NO: 4;
3—with an expression cassette with EGFP transgene under control of a promoter corresponding to the nucleic sequence of SEQ ID NO: 5;
4—with an expression cassette with EGFP transgene under control of a promoter corresponding to the nucleic sequence of SEQ ID NO: 6;
5—with an expression cassette with EGFP transgene under control of a promoter corresponding to the nucleic sequence of SEQ ID NO: 7;
6—with an expression cassette with EGFP transgene under control of a promoter corresponding to the nucleic sequence of SEQ ID NO: 13;
7—with an expression cassette with EGFP transgene under control of a promoter corresponding to the nucleic sequence of SEQ ID NO: 14;
8—with an expression cassette with EGFP transgene under control of a promoter corresponding to the nucleic sequence of SEQ ID NO: 11;
9—with an expression cassette with EGFP transgene under control of a promoter corresponding to the nucleic sequence of SEQ ID NO: 12;
10—with an expression cassette with EGFP transgene under control of a promoter corresponding to the nucleic sequence of SEQ ID NO: 3.

FIG. 13 is a graph of measurement of tissue specificity of produced sequences on the Huh7, HepG2 and HEK293 cell lines, which shows increased intensity of fluorescence of cells expressing the EGFP reporter protein under control of promoters corresponding to the nucleic sequences of SEQ ID NO: 7 and 11. Intensity of fluorescence of cells expressing EGFP following transfection:
1—with an expression cassette with EGFP transgene and without a promoter region (control);
2—with an expression cassette with EGFP transgene under control of a promoter corresponding to the nucleic sequence of SEQ ID NO: 7;
3—with an expression cassette with EGFP transgene under control of a promoter corresponding to the nucleic sequence of SEQ ID NO: 11.

FIG. 14 is a graph that shows increased levels of production of coagulation protein while delivery of an expression cassette comprising a coagulation factor sequence under control of promoter regions corresponding to SEQ ID NO: 3, 8, 11 and 14, in the form of an AAV-based expression vector.

Levels of production of the coagulation protein while transduction:
1—with an expression vector comprising a cassette with coagulation factor transgene and without a promoter region (control);
2—with an expression vector comprising a cassette in which the coagulation factor transgene is under control of a promoter corresponding to the nucleic sequence of SEQ ID NO: 8;
3—with an expression vector comprising a cassette in which the coagulation factor transgene is under control of a promoter corresponding to the nucleic sequence of SEQ ID NO: 11;
4—with an expression vector comprising a cassette in which the coagulation factor transgene is under control of a promoter corresponding to the nucleic sequence of SEQ ID NO: 14;
5—with an expression vector comprising a cassette in which the coagulation factor transgene is under control of a promoter corresponding to the nucleic sequence of SEQ ID NO: 3;

FIG. 15 is a graph that shows increased levels of activity of coagulation protein while delivery of an expression cassette comprising a coagulation factor sequence under control of promoter regions corresponding to SEQ ID NO: 3, 8, 11 and 14, in the form of an AAV-based expression vector.

Levels of activity of the coagulation protein while transduction:
1—with an expression vector comprising a cassette with coagulation factor transgene and without a promoter region (control);
2—with an expression vector comprising a cassette in which the coagulation factor transgene is under control of a promoter corresponding to the nucleic sequence of SEQ ID NO: 8;
3—with an expression vector comprising a cassette in which the coagulation factor transgene is under control of a promoter corresponding to the nucleic sequence of SEQ ID NO: 11;
4—with an expression vector comprising a cassette in which the coagulation factor transgene is under control of a promoter corresponding to the nucleic sequence of SEQ ID NO: 14;
5—with an expression vector comprising a cassette in which the coagulation factor transgene is under control of a promoter corresponding to the nucleic sequence of SEQ ID NO: 3;

FIG. 16 is a graph that shows increased levels of production of coagulation protein while delivery of an expression cassette comprising a coagulation factor sequence under control of promoter regions corresponding to SEQ ID NO: 8 and 11, in the form of a rAAV5 or rAAV6-based expression vector.

Levels of production of the coagulation protein while transduction:
1—with an expression vector comprising a cassette with coagulation factor transgene and without a promoter region (control);
2—with an expression vector comprising a cassette in which the coagulation factor transgene is under control of a promoter corresponding to the nucleic sequence of SEQ ID NO: 11;
3—with an expression vector comprising a cassette in which the coagulation factor transgene is under control of a promoter corresponding to the nucleic sequence of SEQ ID NO: 8.

FIG. 17 is a graph that shows increased levels of activity of coagulation protein while delivery of an expression cassette comprising a coagulation factor sequence under control of promoter regions corresponding to SEQ ID NO: 8 and 11, in the form of a rAAV5 or rAAV6-based expression vector.

Levels of activity of the coagulation protein while transduction:
1—with an expression vector comprising a cassette with coagulation factor transgene and without a promoter region (control);
2—with an expression vector comprising a cassette in which the coagulation factor transgene is under control of a promoter corresponding to the nucleic sequence of SEQ ID NO: 11;
3—with an expression vector comprising a cassette in which the coagulation factor transgene is under control of a promoter corresponding to the nucleic sequence of SEQ ID NO: 8.

FIG. 18 is a graph that shows increased levels of the coagulation factor protein under control of promoter regions corresponding to the nucleic acids of SEQ ID NO: 8 and 11 when delivered as an AAV-based expression vector in vivo to B6.129S-F8tm1Smoc mice.

Levels of coagulation factor protein in animal plasma following injection:
1-control solution without AAV (negative control).
2—with an AAV-based expression vector comprising a cassette in which the coagulation factor transgene is under control of a promoter corresponding to the nucleic sequence of SEQ ID NO: 8.
3—with an AAV-based expression vector comprising a cassette in which the coagulation factor transgene is under control of a promoter corresponding to the nucleic sequence of SEQ ID NO: 11.

FIG. 19 is a graph that shows increased levels of activity of the coagulation factor under control of promoter regions corresponding to the nucleic acids of SEQ ID NO: 8 and 11 when delivered as an AAV-based expression vector in vivo to B6.129S-F8tm1Smoc mice.

Levels of coagulation factor protein in animal plasma following injection:
1-control solution without AAV (negative control).
2—with an AAV-based expression vector comprising a cassette in which the coagulation factor transgene is under control of a promoter corresponding to the nucleic sequence of SEQ ID NO: 8.
3—with an AAV-based expression vector comprising a cassette in which the coagulation factor transgene is under control of a promoter corresponding to the nucleic sequence of SEQ ID NO: 11.

### EXAMPLES

The following examples are provided for better understanding of the invention. These examples are for purposes of illustration only and are not to be construed as limiting the scope of the invention in any manner.

All publications, patents, and patent applications cited in this specification are incorporated herein by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended embodiments.

### Materials and General Methods

### Recombinant DNA Techniques

Standard methods were used to manipulate DNA as described in Sambrook, J. et al, Molecular cloning: A laboratory manual; Cold Spring Harbor Laboratory Press, Cold Spring Harbor, New York, 2012. The molecular biological reagents were used according to the manufacturer protocols. Briefly, plasmid DNA was produced for further manipulation in E. coli cells grown under selective antibiotic pressure so that the plasmids were not lost in the cell population. We isolated the plasmid DNA from cells using commercial kits, measured the concentration, and used it for cloning by restriction endonuclease treatment or PCR amplification. The DNA fragments were ligated to each other using ligases and transformed into bacterial cells for the selection of clones and further production. All resulting genetic constructs were confirmed by restriction patterns and complete Sanger sequencing.

### DNA Sequence Determination

DNA sequences were determined by Sanger sequencing. DNA and protein sequences were analyzed and sequence data was processed in SnapGene Viewer 4.2 or higher for sequence creation, mapping, analysis, annotation and illustration.

### Culturing Cell Cultures

The experiments used HEK293 (Human Embryonic Kidney clone 293 cell lines), HUH7 (human hepatocellular carcinoma cell lines), U87-MG (Uppsala 87 Malignant Glioma cell lines), CHO-K1-S (Chinese hamster ovary cell lines) and HepG2 (human hepatocellular carcinoma cell lines). The suspended HEK293 cells used to produce AAV were cultured under standard conditions at 37° C. and 5% CO2 on a complete culture medium without FBS and antibiotic. The adherent HEK293, HUH7 and HepG2 cells used to test the efficacy of expression of EGFP and AAV products were cultured under standard conditions at 37° C. and 5% CO2, on a complete DMEM medium supplemented with 10% FBS, antibiotic/antimycotic. Adhesive U87-MG and CHO-K1-S cells used for tissue specificity testing were cultured under standard conditions at 37° C. and 5% CO2 on a complete EMEM nutrient medium supplemented with 10% FBS, antibiotic/antimycotic, and DMEM/F12 medium supplemented with 5% FBS, antibiotic/antimycotic, respectively.

Cells were subcultured upon reaching 80-90% confluence. TrypLE Select enzyme (10×) was used to dissociate the cell monolayer. Cell viability was assessed using Trypan Blue stain and disposable cell counting chambers using an automatic Countess II counter.

### Transfection of Cell Cultures

To assess the functional activity of new promoter variants while transfection, we used plasmids comprising an expression cassette for expression of various variants of transgenes of EGFP and coagulation factor. The model cell lines were pre-seeded into the wells of 12-well plates at a density of 10000 cells/cm². A day later, we added an equal copy number of plasmid DNA of the test and control samples as part of a complex with Lipofectamine 3000. For the variants of transgene based on the gene encoding green fluorescent protein (EGFP), on day 3 following transfection, we determined the proportion of EGFP-expressing cells and the intensity of cell fluorescence by flow cytometry. For the variant of transgene based on the coagulation factor, on day 7 following transfection, we determined the content of coagulation protein and activity thereof in the culture medium by ELISA and chromogenic assay. Intact model line cells were used as a negative control.

### Generation and Purification of Viral Particles of AAV Recombinant Vectors

To produce recombinant AAV viral particles comprising a coagulation factor-based transgene, we used HEK293 producer cells which were transfected with 3 plasmids:
A plasmid comprising an AAV expression cassette for expression of coagulation factor;
A plasmid comprising Cap genes of serotype AAV6/AAV5 and Rep genes of serotype AAV2. Each gene, using alternative reading frames, encodes several protein products;
A plasmid comprising Ad2 adenovirus genes required for assembly and packaging of AAV capsids.

72 hours following transfection, the cells were lysed and then the viral particles were purified and concentrated using filtration, chromatography and ultracentrifugation methods. The titer of the viral particles was determined by quantitative PCR with primers and a sample that were specific for the region of the recombinant viral genome and expressed as the copy number of viral genomes per 1 ml.

### Transduction of Cell Cultures

The HUH7 cell line was pre-seeded into the wells of 12-well plates at a density of 10,000 cells/cm². After the cells were attached to the adhesive substrate, AAV preparations were introduced at MOI of 500,000 vg/cell. On day 7 following transduction, the level and activity of the coagulation protein in the culture fluid were determined by ELISA and chromogenic assay. Studies involving the assessment of the level and activity of the coagulation protein in the culture fluid were performed in 6 independent experiments. Intact cells were used as a negative control.

### Determination of Proportion of Cells Expressing EGFP Reporter Protein and Intensity of Cell Fluorescence Using Flow Cytometry

To assess the expression of the EGFP reporter protein on a flow cytofluorometer, on day 3 following transfection, a pre-prepared mixture of buffer and propidium iodide (PI) was added to the cell precipitates at a rate of 1 μl of PI at a concentration of 10 μg/ml per 1 ml of buffer. Cells unstained with PI and non-transfected with plasmid DNA (isotype) were transferred to the wells of a 96-well microplate to assess compensation, a buffer/PI mixture was added to the remaining wells of the microplate to measure controls with PI and the samples. After transferring the cell samples to the microplate, they were incubated for 5 min in the absence of light. After incubation, the microplates with the test cell samples were loaded into a flow cytometer for analysis. Flow cytometry was used to determine the percentage of cells comprising EGFP, as well as the average intensity of fluorescence signal. Each sample was measured in three technical replicates (the number of analyzed events is not less than 10,000).

### Determination of Amount of Coagulation Factor Protein by ELISA

The content of the coagulation factor protein in culture fluid following HUH7 cell transfection and transduction with target candidates was assessed by sandwich method of non-competitive solid-phase enzyme immunoassay (ELISA). Briefly, culture fluid samples diluted in a commercial buffer were transferred to a 96-well microplate sensitized with primary coagulation factor-specific antibodies. The same microplate was loaded with standards for plotting a calibration curve, controls. The plate was incubated for 1 hour at a temperature of 37° C. The microplate wells were washed with buffer prior to introducing biotinylated antibodies, horseradish peroxidase conjugated streptavidin solution, and tetramethylbenzidine (TMB) substrate. Next, a solution with biotinylated detecting coagulation factor-specific antibodies was introduced and the microplate was incubated for 30 minutes at a temperature of 37° C. Streptavidin horseradish peroxidase conjugate solution was added to the resulting complex, and the plate was incubated for 30 minutes at a temperature of 37° C. TMB solution was introduced to visualize the enzyme reaction. Upon achieving the required degree of staining intensity, a stop solution was added to all wells to stop the reaction. After stopping the reaction, the optical density was measured. The concentration of coagulation factor in test samples was measured by way of normalization of chromogenic staining by the standard sample calibration curve, taking into account the pre-dilution factor.

### Determination of Activity Level of Coagulation Factor Protein by ELISA

The activity of the coagulation factor protein in culture medium following HUH7 cell transfection and transduction with target candidates and control samples was assessed using a chromogenic assay. The assay is based on the fact that in the presence of calcium ions, phospholipids and factor IXa, factor X transforms into the activated form Xa, factor VIII functions as a cofactor in the reaction, and the rate of factor X activation is linearly associated with the level of factor VIII. Briefly, the culture medium samples diluted in a commercial buffer, standards for plotting a calibration curve and controls were transferred to a 96-well microplate. Next, the samples were subjected to incubation for 3 minutes at a temperature of 37° C., followed by introduction, into the microplate wells, of the Factor reagent solution comprising factor IXa, factor X, thrombin, CaCl₂ and phospholipids. Thereafter, the microplate was incubated for 4 minutes at a temperature of 37° C., followed by introduction of chromogenic substrate solution S-2765+1-2581 into the wells. The plate was then incubated for 7 minutes at a temperature of 37° C. Upon achieving the required degree of staining intensity, a 20% solution of acetic acid was added to all wells to stop the reaction. The optical density of the solutions in the microplate wells was then measured. The activity of coagulation factor in test samples was measured by way of normalization of chromogenic staining by the standard sample calibration curve, taking into account preliminary dilution

### In Vivo Study on Laboratory Animals

B6.129S-F8tm1Smoc mice deficient of coagulation factor (males aged 6-8 weeks) were used for experiments. The products were administered to animals by way of a single intravenous injection into the tail vein. A buffer solution free of AAV was administered into the negative control group of animals. Blood plasma sampling was performed on the day of injection before administering the products, then on day 70 following introducing the expression vectors. All animal tests were conducted in full compliance with the ARRIVE guidelines

### Statistical Data Analysis

The results indicate an average value #standard deviation (SD), one-way analysis of variance (ANOVA) followed by Dunnett's multiple pairwise comparisons was employed to compare the experiment results, and they were determined to be statistically significant.

### Example 1

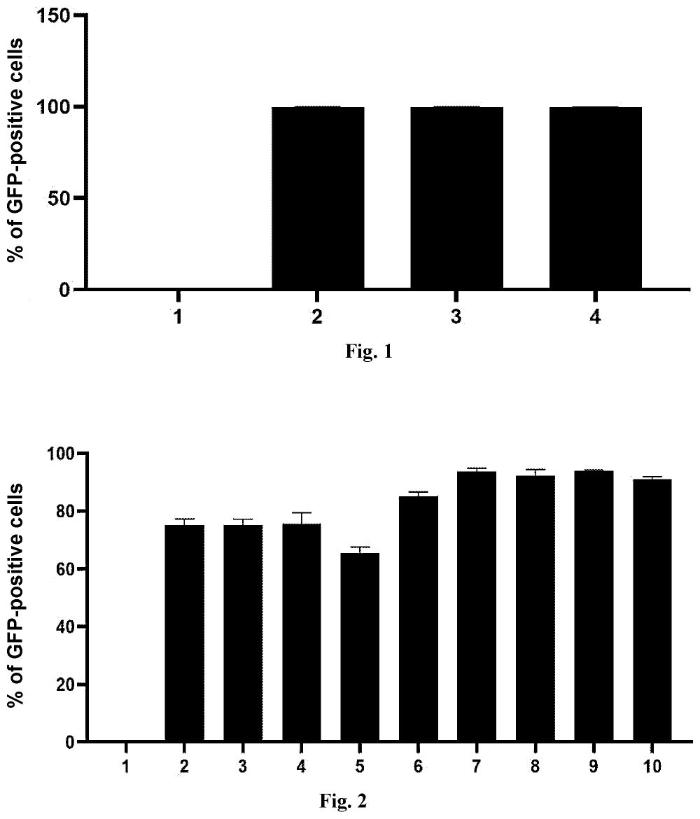

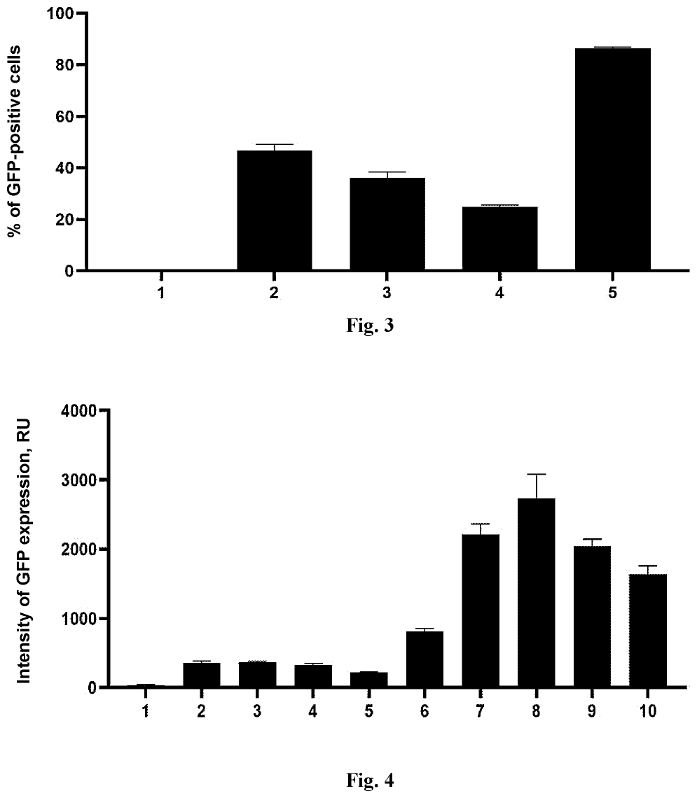

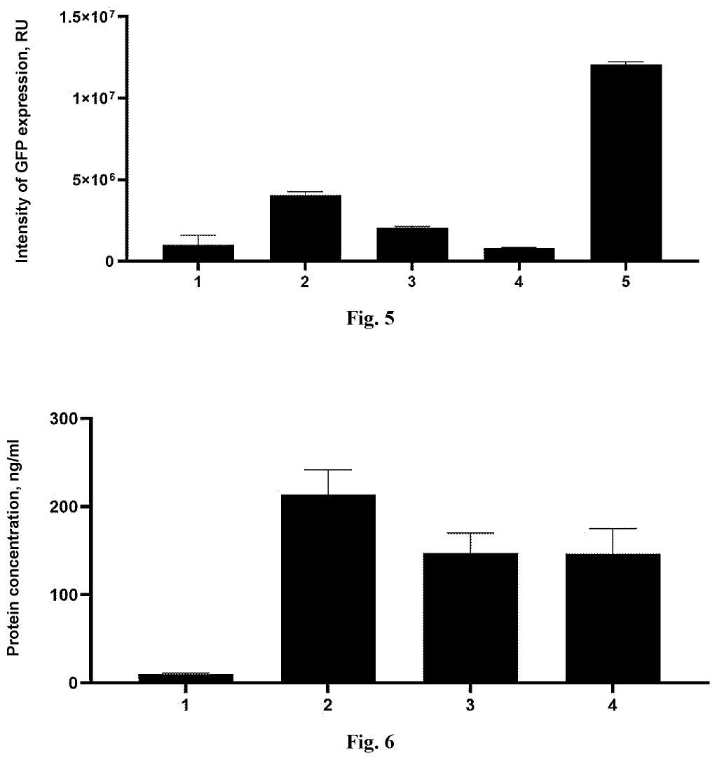

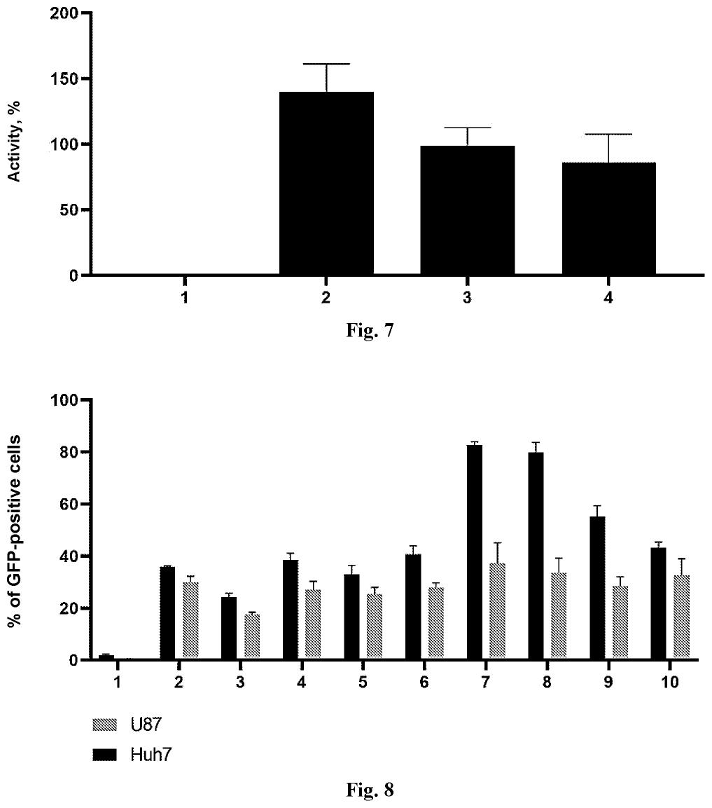

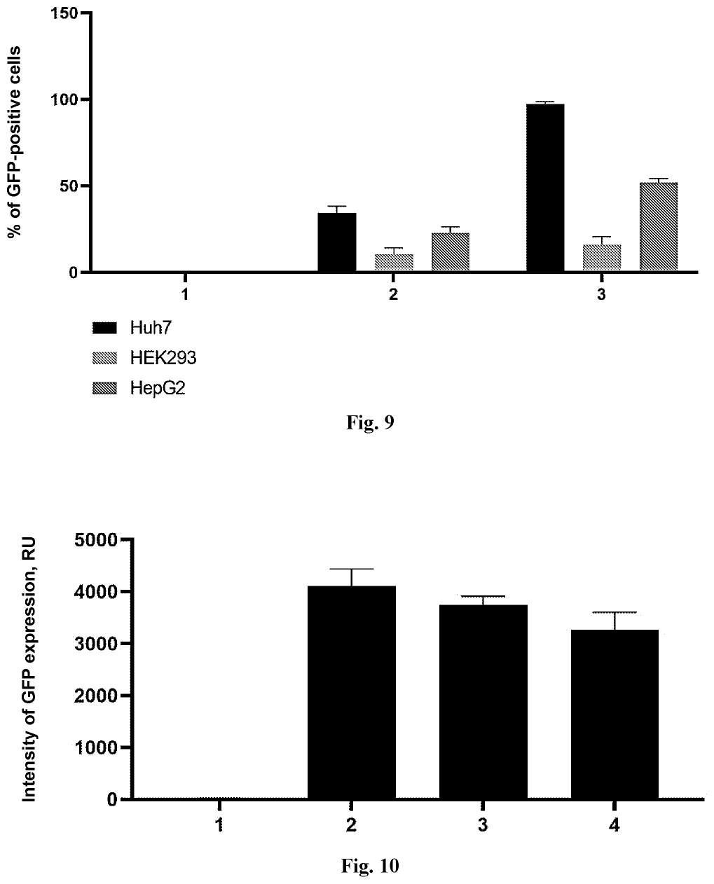

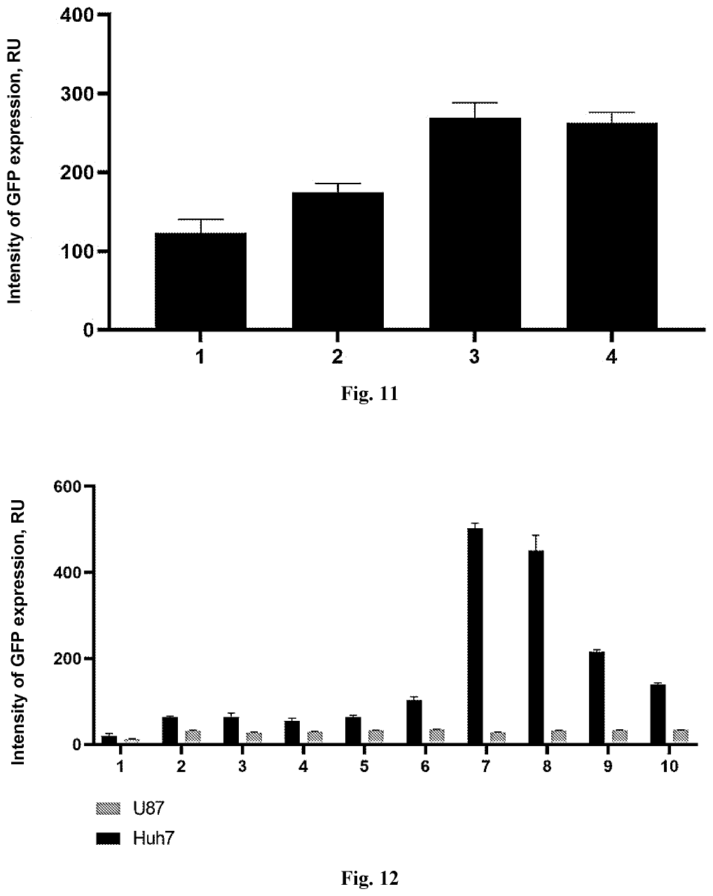

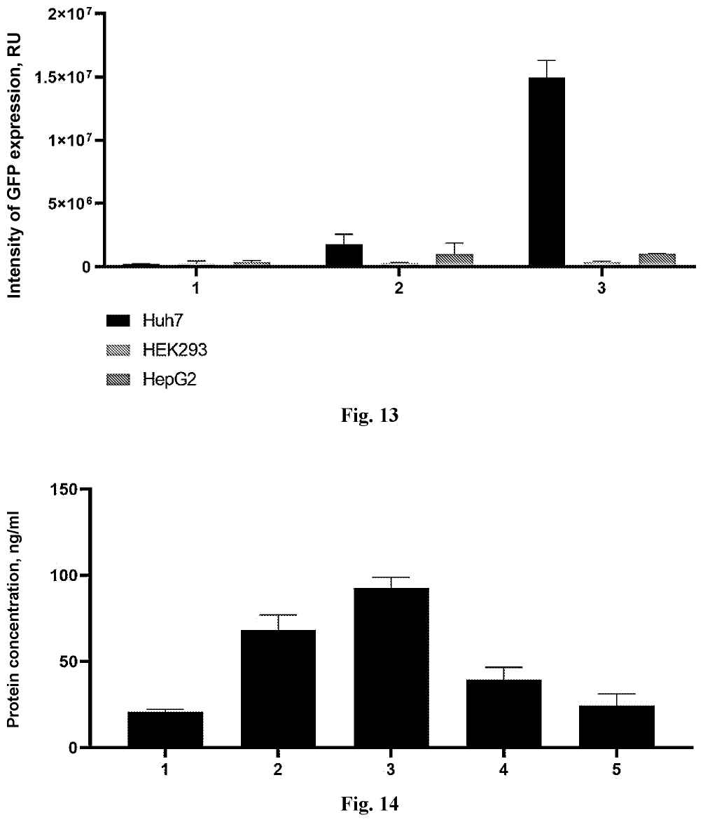

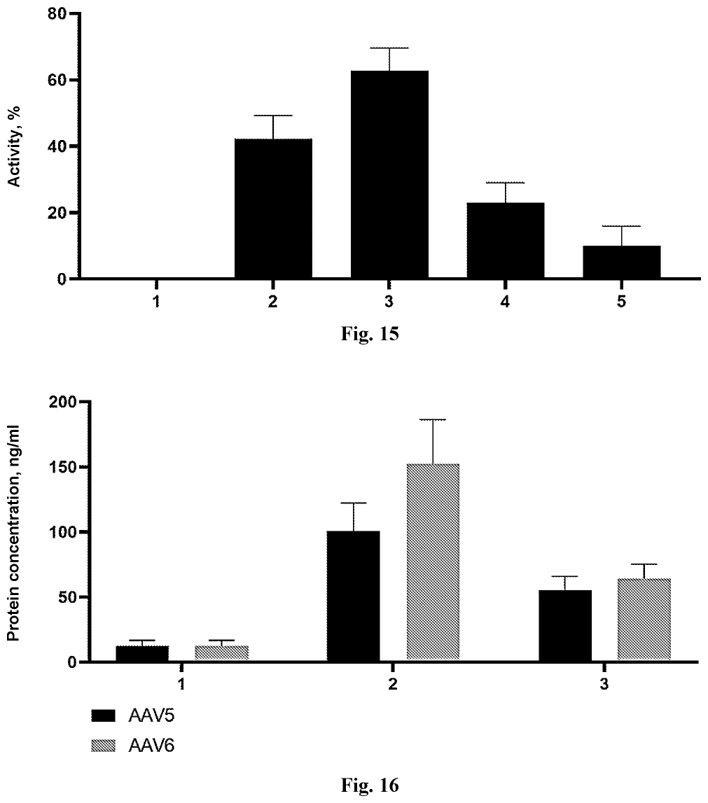

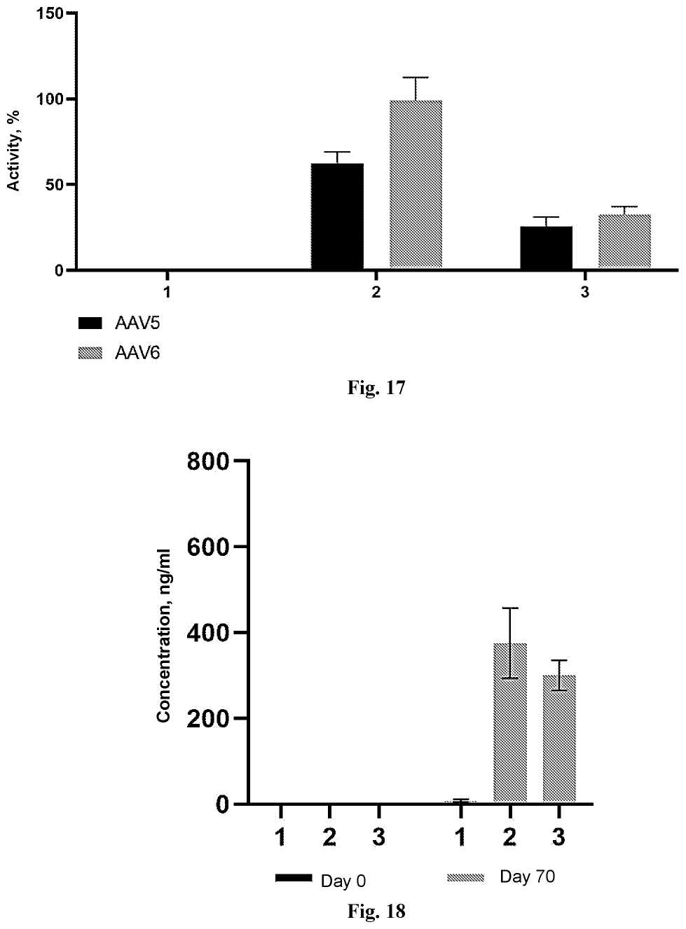

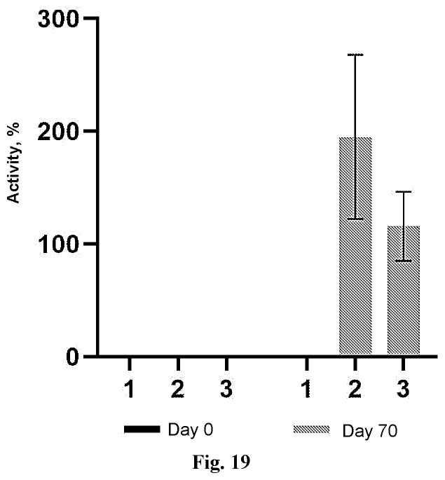

In order to increase levels of expression of the gene of interest, while designing genetic constructs we used a panel of promoters (SEQ ID NO: 1, 2, 3, 4, 5, 6, 7, 8, 9, 10, 11, 12, 13, 14 or 15) which were developed based on transcription factor binding sites. These nucleotide sequences as part of an expression cassette consisting of a left-hand (first) ITR (inverted terminal repeats) corresponding to the sequence of SEQ ID NO: 16, a promoter selected from a group comprising: SEQ ID NO: 1, 2, 3, 4, 5, 6, 7, 8, 9, 10, 11, 12, 13, 14 or 15, the gene of interest, a polyadenylation signal (SEQ ID NO: 17), a right-hand (second) ITR (SEQ ID NO: 18), wherein the gene of interest may be a green fluorescent protein (GFP) sequence or coagulation cascade protein sequence were tested in vitro by way of transfecting model cell lines (HEK293, HUH7, U87-MG, CHO-K1-S and HepG2). An expression cassette comprising a left-hand (first) ITR (SEQ ID NO: 16), the gene of interest, a polyadenylation signal (SEQ ID NO: 17), a right-hand (second) ITR (SEQ ID NO: 18) was used as a control. FIGS. 1 and 2 also show additional controls comprising the same elements of the expression cassette supplemented with a constitutive cytomegalovirus (CMV) viral promoter.

All nucleic acids of SEQ ID NO:1, 2, 3, 4, 5, 6, 7, 8, 9, 10, 11, 12, 13, 14 or 15, which are promoter regions, provide for an increased proportion (FIG. 1-3) of cells expressing the EGFP reporter protein and increased intensity of EGFP fluorescence (FIG. 4-5) when the green fluorescent protein sequence is used as the gene of interest. When the coagulation factor sequence was used as the gene of interest, we also observed increased levels of production (FIG. 6) and activity (FIG. 7) of the coagulation protein as compared to the use of the nucleic acid of the gene of interest without a promoter region. Thus, the nucleic acids (SEQ ID NO: 1-15) exhibit promoter activity as part of an expression cassette with various variants of transgenes while transfection of model cell lines (Huh7, CHO-K1-S and HepG2).

### Example 2

In order to measure the tissue specificity of the developed nucleic acid sequences (SEQ ID NO:1, 2, 3, 4, 5, 6, 7, 8, 9, 10, 11, 12, 13, 14 or 15), which have promoter activity, the nucleotide sequences were tested while transfection on model cell lines (HEK293, HUH7, U87-MG, CHO-K1-S and HepG2) in vitro as part of an expression cassette consisting of a left-hand (first) ITR (inverted terminal repeats) corresponding to the sequence of SEQ ID NO: 16, a promoter (SEQ ID NO: 1-15), the gene of interest, a polyadenylation signal (SEQ ID NO: 17), a right-hand (second) ITR (SEQ ID NO: 18), wherein the gene of interest may be represented by an EGFP protein sequence or nucleic acids encoding the coagulation cascade proteins. An expression cassette comprising a left-hand (first) ITR (SEQ ID NO: 16), the gene of interest, a polyadenylation signal (SEQ ID NO: 17), a right-hand (second) ITR (SEQ ID NO: 18) was used as a control. Also, in FIGS. 8, 10, 11 and 12 there was used a control comprising the same elements of the expression cassette supplemented with cytomegalovirus (CMV) viral promoter region.

All nucleic acids of SEQ ID NO:1, 2, 3, 4, 5, 6, 7, 8, 9, 10, 11, 12, 13, 14 or 15, which are promoter regions, provide for increased proportion (FIGS. 8 and 9) of cells expressing the EGFP reporter protein, and increased intensity of EGFP fluorescence (FIG. 10-13) in cell lines of hepatocytic origin as compared to the use of a nucleic acid without a promoter region. Thus, the nucleic acids (SEQ ID NO: 1, 2, 3, 4, 5, 6, 7, 8, 9, 10, 11, 12, 13, 14 or 15) have tissue-specific promoter activity in cells of hepatocytic origin.

### Example 3

We produced AAV-based expression vectors comprising expression cassettes encoding a transgene (coagulation factor) under control of the nucleic acids of SEQ ID NO: 8, 11 and 14 which have promoter activity. Said expression vectors were checked by transducing Huh7 cells in vitro. An AAV-based expression vector free of a promoter region was used as a control.

The use of expression vectors comprising the nucleic acids of SEQ ID NO: 8, 11 and 14 leads to increased levels of production (FIG. 14) and activity (FIG. 15) of the coagulation protein as compared to the use of the expression vector free of a promoter region. Use of the expression vectors based on AAV of various serotypes comprising the nucleic acids of SEQ ID NO: 8 and 11 exhibited increased levels of production (FIG. 16) and activity (FIG. 17) of the coagulation protein as compared to those of the control. The results correspond to the data obtained while transfection of Huh7 cells (FIGS. 6 and 7). Thus, the nucleic acids (SEQ ID NO:1, 2, 3, 4, 5, 6, 7, 8, 9, 10, 11, 12, 13, 14 or 15) exhibit promoter activity while being delivered as an expression vector with various variants of transgenes while transduction of model cell lines.

### Example 4

In order to measure the promoter activity of nucleic acids of SEQ ID NO: 8 and 11 as part of the expression vector in vivo, we produced AAV-based products where the transgene is a coagulation factor under control of the nucleic acids of SEQ ID NO: 8 or 11. Next, the test products were administered to B6.129S-F8tm1Smoc laboratory mice. A buffer for dilution of the viral product, free of AAV particles was used as a negative control. The AAV products were administered to animals once by way of intravenous hydrodynamic injection into the tail vein. Blood plasma sampling was performed on the day of injection before administering the products (day 0), then on day 70 following administering.

In vivo studies showed that in the case of using the test drugs comprising the coagulation factor gene sequence under control of the nucleic acids of SEQ ID NO: 8 and 11 there was observed a significantly increased amount of coagulation factor and activity thereof in animals' plasma on day 70 following administering the products (FIG. 18-19). Thus, the nucleic acids of SEQ ID NO: 8 and 11 exhibit promoter activity when delivered as an AAV-based expression vector in vivo.

1. A nucleic acid having promoter activity, comprising a nucleotide sequence selected from the group comprising SEQ ID NO:1, 2, 3, 4, 5, 6, 7, 8, 9, 10, 11, 12, 13, 14 or 15.

2. An expression cassette comprising a molecule of the nucleic acid according to claim 1 and an operably linked transgene.

3. The expression cassette according to claim 2, wherein the transgene encodes a protein or a small inhibitory nucleic acid.

4. The expression cassette according to claim 3, wherein the transgene encodes a protein that is selected from the group comprising factor VIII or a functional variant thereof, factor IX or a functional variant thereof, SMN1 protein (survival motor neuron protein), the RBD-S polypeptide of SARS-cov2, or a therapeutic antibody.

5. An expression vector comprising the nucleic acid according to claim 1 or the expression cassette according to any one of claims 2 to 4.

6. The expression vector according to claim 5, which is a plasmid, AAV, lentivirus or adenovirus.

7. The expression vector according to claim 6, which is an AAV selected from the group comprising the following AAV serotypes: AAV1, AAV2, AAV3, AAV4, AAV5, AAV6, AAV7, AAV8, AAV9, AAV10 AAV11, AAV12, AAV13, AAV14, AAV15, AAV16, rAAV.rh8, rAAV.rhlO, rAAV.rh20, rAAV.rh39, rAAV.Rh74, rAAV.RHM4-1, AAV.hu37, rAAV.Anc80, rAAV.Anc80L65, rAAV.7m8, rAAV.PHP.B, rAAV2.5, rAAV2.6, rAAV2.8, rAAV2.9, rAAV2tYF, rAAV3B, rAAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15 or AAV.HSC16.

8. A host cell for producing the target product or expression vector according to claims 5 to 7, comprising the nucleic acid according to claim 1 or the expression cassette according to any one of claims 2 to 4.


SEQUENCE LISTING (18 sequences)

SEQ ID NO: 1 - DNA, 201 bp, synthetic construct
  source 1..201: mol_type = other DNA
tgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacag

SEQ ID NO: 2 - DNA, 209 bp, synthetic construct
  source 1..209: mol_type = other DNA
tgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgt

SEQ ID NO: 3 - DNA, 168 bp, synthetic construct
  source 1..168: mol_type = other DNA
ttgcaatgtttgcccattttacccagccagtggacttagcccctgtttgctcctccgataactggggtgaccttggttaatattcaccagcagcctcccccgttgcccctctggatccactgcttaaatacggacgaggacagggccctgtctcctcagcttcaggca

SEQ ID NO: 4 - DNA, 158 bp, synthetic construct
  source 1..158: mol_type = other DNA
ttgcaatgtttgcccattttgtggtttccccagccagtggacttagcccctgtttgctcctccgataactggggtgaccttggttaatattcaccagcagcctcccccgttgcccctctggatccactgcttaaatacggacgaggacagggccctgt

SEQ ID NO: 5 - DNA, 154 bp, synthetic construct
  source 1..154: mol_type = other DNA
ttgcaatgtttgcccattttactgagccaggggcccagccagtggacttagcccctgtttgctcctccgataactggggtgaccttggttaatattcaccagcagttgcccctctggatccactgcttaaatacggacgaggacagggccctgt

SEQ ID NO: 6 - DNA, 150 bp, synthetic construct
  source 1..150: mol_type = other DNA
ttgcaatgtttgcccattttagtggtttccccagccagtggacttagcccctgtttgctcctccgataactggggtgaccttggttaatattcaccagcagttgcccctctggatccactgcttaaatacggacgaggacagggccctgt

SEQ ID NO: 7 - DNA, 123 bp, synthetic construct
  source 1..123: mol_type = other DNA
ttgcaatgtttgcccattttgtggtttagccagtggacttagcccctgtttgctcctccgataactggggtgaccttggttaatattcaccctgcttaaatacggacgaggacagggccctgt

SEQ ID NO: 8 - DNA, 127 bp, synthetic construct
  source 1..127: mol_type = other DNA
gcccaaggtcttacataagaggacgtggtttagccagtggacttagcccctgtttgctcctccgataactggggtgaccttggttaatattcaccctgcttaaatacggacgaggacagggccctgt

SEQ ID NO: 9 - DNA, 123 bp, synthetic construct
  source 1..123: mol_type = other DNA
agttaatgattaactattttgtggtttagcctccaaagtccaaaatgtaaacatactccgataactggggtgaccttggttaataattaacctgcttaaatacggacgaggacagggccctgt

SEQ ID NO: 10 - DNA, 136 bp, synthetic construct
  source 1..136: mol_type = other DNA
agttaatgattaactattttgtggtttagcctccaaagtccaaaatgtaaacatactccgataactggggtgaccttggttaataattaacgttaataattttcctgcttaaatacggacgaggacagggccctgt

SEQ ID NO: 11 - DNA, 176 bp, synthetic construct
  source 1..176: mol_type = other DNA
ttgcaatgtttgcccattttacagccagtggacttagcccctgtttgctcctccgataactggggtgaccttggttaatattcaccagcagttaataattttctcccccgttgcccctctggatccactgcttaaatacggacgaggacagggccctgtctcctcagcttcaggca

SEQ ID NO: 12 - DNA, 170 bp, synthetic construct
  source 1..170: mol_type = other DNA
ttgcaatgtttgccccagccagtggacttagcccctgtttgctcctccgataactggggtgaccttggttaatattcaccagcagttaataattttctcccccgttgcccctctggatccactgcttaaatacggacgaggacagggccctgtctcctcagcttcaggca

SEQ ID NO: 13 - DNA, 163 bp, synthetic construct
  source 1..163: mol_type = other DNA
ttgcaatgtttgcccccagtggacttagcccctgtttgctcctccgataactggggtgaccttggttaatattcaccgttaataattttctcccccgttgcccctctggatccactgcttaaatacggacgaggacagggccctgtctcctcagcttcaggca

SEQ ID NO: 14 - DNA, 252 bp, synthetic construct
  source 1..252: mol_type = other DNA
tgtttgctgcagttaatgattaactattttagggtggacacaggacgctgtggtttctgagccagggggcgactcagatcccagcctccaaagtccaaaatgtaaacatactccgataactggggtgaccttggttaataattaacagcagcctcccccgttgcccctctggatccactgcttaaatacggacgaggacagggccctgtctcctcagcttcaggcaccaccactgacctgggacagtgaatc

SEQ ID NO: 15 - DNA, 176 bp, synthetic construct
  source 1..176: mol_type = other DNA
agttaatgattaactattttacagcctccaaagtccaaaatgtaaacatactccgataactggggtgaccttggttaataattaacagcagttaataattttctcccccgttgcccctctggatccactgcttaaatacggacgaggacagggccctgtctcctcagcttcaggca

SEQ ID NO: 16 - DNA, 130 bp, synthetic construct
  source 1..130: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 17 - DNA, 60 bp, synthetic construct
  source 1..60: mol_type = other DNA
cgcgaataaaagatctttattttcattagatctgtgtgttggttttttgtgtgatgcagc

SEQ ID NO: 18 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg